(19) United States
(12) Patent Application Publication
(10) Pub. No.: US 2012/0231117 A1
(43) Pub. Date: Sep. 13, 2012

(54) FOOD PRODUCTS HAVING IMPROVED HEAT STABILITY

(76) Inventors: Christina Josephina Antonia Maria Timmer-Keetels, Witteveen (NL); Johannes Andries Nieuwenhuijse, Eefde (NL); Johanna Henriette Zijtveld-van der Wiel, Baarn (NL)

(21) Appl. No.: 13/395,759

(22) PCT Filed: Sep. 14, 2010
(86) PCT No.: PCT/NL2010/050585

§ 371 Date: May 17, 2012

(30) Foreign Application Priority Data
Sep. 15, 2009 (NL) 2003494

(51) Int. Cl. A23J 1/20; A23C 9/20; A23C 13/14; A23C 9/18; C07K 14/47; A23C 9/12

(52) U.S. Cl. 426/42; 530/350; 426/580; 426/586; 426/587; 426/588

(57) ABSTRACT

The invention relates to a process for the production of a food product comprising milk protein. Provided is a process for the production of a food product comprising a milk protein, comprising subjecting said milk protein to an enzymatic deamidation procedure, formulating the deamidated milk protein into a liquid food product, followed by either heat sterilization of the food product or concentration and spray drying of the food product into a powder. Also provided are products obtainable by the method.

The invention relates to a process for the production of a food product comprising milk protein. In particular, it relates to a food product having improved heat stability and/or increased nutritional value.

In the food industry, heat sterilization is commonly applied as to obtain safe products with a satisfactory shelf life. However, products do not always withstand such intense heat treatments. They may for instance aggregate or coagulate upon heating, which can lead to an undesirable consistency or appearance of the product. The resistance to heat treatments is often referred to as the heat stability of the product.

Liquid food products comprising milk proteins, which can for example be emulsified and/or concentrated food products, such as infant food, evaporated milk, clinical nutritional formulas, and creamers, often suffer from insufficient heat stability. In order to be able to produce shelf stable products with a satisfactory consistency or appearance, it may involve the incorporation of undesirable heat stabilizing agents such as phosphate salts to the products. Insufficient heat stability may also limit the scope within which the formulation of the food product can be adapted, such as with respect to the protein and/or mineral content. In general, a complex system of factors determines whether a product comprising milk protein can withstand heat sterilization. Even the relation between a single factor such as the pH of the product and heat stability can already be very fanciful. Often, extensive research effort is required to assess suitable process conditions and product compositions in order to be able to produce heat sterilized liquid food products comprising milk proteins.

Infant food can be an emulsified food product that often largely consists of dairy ingredients such as skim milk, whey proteins, lactose and/or caseinate. Ingredients from a non-dairy source may be added in order to make the infant food more suitable for human beings. This involves addition of ingredients such as carbohydrates, minerals, vitamins and fats. Heat stability is a major issue in the production of infant food, limiting the scope within which the composition of the products can be adapted to better satisfy the nutritional needs of babies and young children. It is widely known that, for example, the dilution of milk in infant food or the addition of whey proteins or minerals to infant food can lead to problems with the heat stability (McSweeney, Food Hydrocolloids 18 (2004) 109-125).

Clinical nutritional formulas are products for ill people, people with a poor physical condition or other disabilities that restrict to have a normal diet or make a normal diet impossible. It comprises oral, enteral and parenteral nutrition. Enteral nutrition is nutrition that is fed in the gastrointestinal tract, often in the form of tube feeding, while parenteral nutrition is fed in the veins of a human being. Oral and enteral nutrition are emulsified products that normally contain proteins in amounts up to 10%. These proteins can be milk proteins such as casein, caseinate, milk protein concentrate, whey and/or milk serum proteins. The products often undergo intense heat sterilization, such as a heating for 20 min at 120° C., in order to make them safe. Heat stability is a major problem, especially when whey and/or milk serum proteins are present in the product.

Insufficient heat stability of the product can also be very disadvantageous with respect to the heat sterilization process itself. The resulting coagulation and/or aggregation and/or other heat-induced behavior of the product can easily lead to fouling and/or clogging of the equipment, which will lead to high cleaning costs and large downtimes. The latter problem is also encountered in the production of dried products comprising milk protein, such as powdered infant or clinical formulas.

It is an object of the invention to provide a process for formulating milk protein into a nutritional product, wherein the above problems are at least partially avoided. In particular, the inventors aimed at providing a liquid food product having improved heat stability. It is a further object of the invention to provide a (liquid) food product which has a more desirable composition, nutritional value, consistency and/or appearance. A still further object of the invention is to provide a liquid food product comprising reduced amounts of heat stabilizing agents and/or infant food with improved mineral and/or protein composition and/or clinical nutritional formula with improved mineral and/or protein composition. It is also a further object of the invention to provide a heating process for liquid food products, involving e.g. heat sterilization, concentration, and/or spray drying, in which less coagulation and/or aggregation and/or other heat-induced behavior of the product occurs, leading to less fouling and clogging of the equipment.

Surprisingly, it has been found that the objects of the invention can be achieved by an enzymatic deamidation treatment of milk protein prior to heating.

The invention thus provides a process for the production of a food product comprising a milk protein, comprising subjecting said milk protein to an enzymatic deamidation procedure, formulating the deamidated milk protein into a liquid food product, followed by either heat sterilization of the food product or concentration and spray drying of the food product into a powder. In one embodiment, the invention provides a method for producing a heat sterilized liquid food product comprising milk protein, comprising subjecting said milk protein to an enzymatic deamidation procedure, formulating the deamidated milk protein into a liquid food product, followed by heat sterilization of the food product. In another embodiment, the invention provides a method for producing a dried or powdered food product comprising milk protein, comprising subjecting said milk protein to an enzymatic deamidation procedure, formulating the deamidated milk protein into a liquid food product, followed by concentration and spray drying of the food product. As will be understood, also the powdered product will be consumed, after reconstitution with a suitable liquid medium such as water, as a liquid food profuct. Methods for preparing powdered products known in the art, see for example Walstra, J. T. M. Wouters & T. J. & Geurts, ‘Dairy Science and Technology’ (Chapter 20, and in particular FIG. 20.1).

A protein deamidating enzyme for use in the present invention acts directly on the amide groups of a protein and can deamidate with neither peptide bond cleavage nor protein crosslinking. Enzymatic deamidation of milk protein can for example be achieved with a protein glutaminase isolated from Chryseobacterium proteolyticum sp No. 6790. This enzyme is able to convert the amide side chain moieties of glutamine into carboxyl groups. Generally, the enzyme does not cleave peptide bonds or crosslink proteins, resulting in a deamidated protein with a molecular weight that is nearly similar to that of the untreated protein. The reaction is illustrated in FIG. 1. Enzymatic deamidation of milk protein can also be achieved with a protein asparaginase, which is able to convert the amide side chain moieties of asparagine into carboxyl groups. As a consequence of the action of the enzyme, the iso-electric point of the protein can decrease. In the present invention, a liquid food product denotes a food product in non-solid and/or non-powder form. In a preferred embodiment, the viscosity of a liquid food product ranges from 1 to 1000 mPa·s, more preferably from 5 to 100 mPa·s at a shear rate of 100 s⁻¹.

The heat treatment of the liquid food product may comprise heat sterilization. It may alternatively comprise concentration and spray drying, for instance if a powdered food product is desired. Heat sterilization includes a heat treatment sufficient to obtain a product with a shelf life of at least 1 month at ambient temperature. This is in contrast to pasteurization, which normally results in a product that is shelf stable at refrigeration temperatures of about 4 to 7° C. only. During heat sterilization virtually all micro-organisms in the product are inactivated. For products with a more neutral pH this can for instance be achieved by heating the product in its package for about 10 to 20 minutes at 120° C. or by an ultra-high temperature (UHT) treatment of about 4 seconds at 140° C. in a flowing condition. Also other time-temperature combinations are possible as a sterilization treatment, and these can be easily assessed by the man skilled in the art.

EP1371734 discloses deamidation of milk protein as a method to denature the protein. According to EP1371734, this can lead to an improvement of the functionality of the proteins in a food product, such as the solubility, dispersibility, foaming performance, foam stability, emulsifiability, and emulsion stability. EP1371734 does not disclose heat sterilization of food products. Also the disclosed liquid food products comprising milk proteins are normally not heat sterilized and do normally not exhibit heat instability.

JP2003250460 also discloses deamidation of milk proteins, in order to provide a milk protein having excellent physicochemical properties such as solubility, viscosity, gelling property, emulsifying property or foaming property, sensory properties such as taste, palatability and flavor or low allergenic property. Similar to EP1371734, no reference is made to heat sterilization. No straightforward relation is known between the functional properties of the protein as disclosed in JP2003250460 and heat stability of food products comprising milk proteins. Moreover, the food products mentioned are usually not heat sterilized and do normally not exhibit heat instability.

EP1839491 relates to a method wherein a protein deamidating enzyme is added to raw milk in order to produce dairy products with smooth oral sensation with suppressed acidic and bitter taste. Particularly, cheese and yoghurt are mentioned. No reference is made to heat sterilization. It is generally known that cheese and yoghurt are normally not heat sterilized and that it even is virtually impossible to heat sterilize these products. Above that, problems with acidic and bitter taste do not generally occur in heat sterilized food products.

WO28138900 discloses a method for the preparation of acidified milk drinks, involving enzymatic deamidation of milk proteins. This can result in acidified milk drinks with fewer tendencies to separate into curd and whey upon storage. No reference is made to heat sterilization, concentration and/or spray drying. Rather, as mentioned in WO28138900, acidified milk drinks are normally heat pasteurized, at temperatures up to 95° C., in order to render them shelf stable. Normally, heat instability does not occur in this type of process.

In a method according to the invention, enzymatic deamidation is preferably carried out with a protein glutaminase as an enzyme. This enzyme is preferably obtained from Chryseobacterium proteolyticum sp No. 6790. The action of the deamidating enzyme does normally neither result in peptide bond cleavage nor in protein crosslinking. Even more preferably, the enzymatic deamidation of the milk protein results in less than 10% change of the average molecular weight of the protein. The molecular weight of the monomer of for example β-lactoglobulin is 18 kDa, of α-lactalbumin is 14 kDa, of β-casein is 24 kDa.

For many food products, proteins from cheese whey, acid whey or milk serum are an important ingredient. Cheese whey is a byproduct of the cheese making process, and remains when cheese curd is separated from milk. Acid whey is a byproduct of for instance caseinate production or cottage cheese production. Milk serum is typically obtained by removal of colloidal particles such as fat globules and casein micelles from milk. This can for instance be done by microfiltration or ultracentrifugation of milk. These whey proteins or milk serum proteins can for example be added because of their specific nutritional and/or texturizing properties. Whey and/or milk serum proteins can for instance be added to infant food, clinical nutritional formulas or sweetened condensed milk. Whey and/or milk serum proteins are especially important ingredients for infant food, because human milk has a higher whey protein/casein ratio than cows milk (and all other dairy milks), so that addition of whey and/or milk serum proteins to infant food can lead to a composition closer to human milk. Also for clinical nutritional formulas, whey and/or milk serum proteins are often considered desirable ingredients, because their specific amino acid composition can provide good nutritional properties. Although the whey proteins or milk serum proteins often make up only a minor part of the total protein amount in food products, it has surprisingly been found that it can be sufficient to carry out an enzymatic deamidation on the whey proteins or milk serum proteins only, e.g. in order to obtain a heat stable liquid food product.

Therefore, in one embodiment, the invention provides a process for the production of a heat sterilized liquid food product comprising a protein from cheese whey, acid whey and/or milk serum, comprising an enzymatic deamidation of said protein from cheese whey, acid whey or milk serum, formulating it into a food product, and heat sterilization of the food product.

Enzymatic deamidation can be carried out by incubating a protein suspension in water with the enzyme. The pH of the protein suspension is preferably between 5 and 8, more preferably between 5.5 and 7.5. The temperature preferably is between 20 and 60° C., more preferably between 30 and 50° C. The enzyme/substrate ratio (E:S) can be as low as 1:100, or even 1:1000. The reaction can be stopped for instance by cooling the protein suspension to a temperature lower than 10° C. or by heating (e.g. 30 minutes at 80° C.) to inactivate the enzyme.

A product resulting from the process as according to the invention preferably concerns an emulsified and/or concentrated product. Emulsified food products according to the invention are products in the form of a dispersion of an oil or fat in water. The oil or fat may be milk fat or fat from another source than milk such as rapeseed, coconut, or palm. Examples of emulsified food products are infant food, products for clinical nutritional formulas, creamers for soup, coffee and other applications. Concentrated food products as according to the invention have been concentrated, for example by means of evaporation or membrane filtration. For dairy products, this can lead to a non-fat dry matter content higher than that of milk. Examples of concentrated dairy products are evaporated milk and sweetened condensed milk. The process as according to the invention preferably comprises the preparation of an emulsion and/or evaporation and/or membrane filtration as formulating steps of a food product. The invention provides a process for the production of a liquid food product, in which the temperature during sterilization is at least 110° C., preferably at least 115° C. The invention also provides a heat sterilized liquid food product obtainable by a process as according to the invention.

Enzymatic deamidation of proteins can lead to the conversion of the amide side chain moieties of glutamine or asparagine into carboxyl groups, resulting in the formation of respectively glutamate or aspartate. A food product of the invention preferably comprises milk protein with glutamate and/or aspartate residues. The heat sterilized liquid food product is for example an infant food, clinical nutritional formula, creamer, evaporated milk or sweetened condensed milk. A dried food product is for instance a powdered infant formula or a powdered clinical formula that is reconstituted before use. Preferably, the invention provides a powdered food product which, when dissolved at 10% (w/v) in an aqueous solvent, has a pH up to 6.8, more preferably up to 6.6. As the unwanted Maillard reaction is favoured by a high pH, a product of the invention has less lysine blockage and increased nutritional value as compared to conventional products.

For infant food, the ratio between serum and/or whey protein on the one hand and casein on the other hand, is generally considered an important factor for the nutritional value of the product. High relative amounts of serum and/or whey protein can be desirable in order to better approach the composition of mother's milk, which is often an important target. In the last decades, the commonly applied casein to whey protein ratio is 50:50 or 40:60. In order to bring the infant formula more close to mother's milk, a lower protein content in infant formula would be desirable. In order to fulfill the amino acid requirements at this lower protein content, a higher ratio of serum and/or whey protein to casein is desirable. These high relative amounts can often not be applied because of problems with heat stability of the products. The use of enzymatically deamidated milk protein can enable the production of infant food with a higher ratio of serum and/or whey protein to casein, such as higher than 0.50/0.50, or even higher than 0.60/0.40 or 0.70/0.30.

For clinical nutritional formula, the fraction of serum and/or whey protein is also generally considered an important factor for the nutritional value of the product, because the use of serum proteins allows optimization of the amino acid composition of the product. However, because of heat stability problems, serum proteins are not applied in all but a few clinical formulas. The use of enzymatically deamidated milk protein can enable the production of clinical nutritional formula with a casein/whey protein ratio of minimal 0.8/0.2, or even minimal 0.7/0.3 or 0.5/0.5.

Infant formula, and some clinical formulas are also available as powders. During production of powder, fouling of equipment should be minimal. Also, Maillard reaction, leading to lysine blockage and thus loss of nutritional value, should be minimal. Solving both problems concomitantly is usually difficult: a common measure to lower fouling is to increase pH, but increasing pH also enhances Maillardation. The use of enzymatically deamidated milk protein increases protein stability and lowers fouling especially at pH below 6.8, and thus allows production of products with less blocked lysine.

For creamer, evaporated milk or sweetened condensed milk, it can be advantageous to have a high ratio of serum and/or whey protein to casein, for instance because this can provide certain functional properties of the product or a lower cost price. Use of such a high ratio of serum and/or whey protein to casein can easily lead to problems with heat stability. The use of enzymatically deamidated milk protein can enable the production of creamer, evaporated milk or sweetened condensed milk with a higher ratio of serum and/or whey protein to casein, such as higher than 0.25/0.75, or even higher than 0.30/0.70 or 0.40/0.60 or 0.50/0.50.

A food product according to the invention can contain various ingredients comprising milk protein. Such ingredients might for instance be skim milk powder, sodium caseinate, potassium caseinate, magnesium caseinate, acid casein, milk protein concentrate, milk serum, milk serum protein concentrate, whey, whey protein concentrate, whey protein isolate, α-lactalbumin, β-lactoglobulin. The protein in at least one of these ingredients has been enzymatically deamidated as to convert at least part of the glutamine and/or asparagine groups into glutamate and/or aspartate. The invention thus provides the use of an enzymatically deamidated milk protein in a heat sterilized liquid or spray dried food product. The milk protein that has been deamidated can for instance on average contain at least 0.1 glutamate and/or aspartate group per monomer of protein, or at least 0.5 or 1.0 glutamate and/or aspartate group per monomer. Preferably the milk protein comprises protein from cheese whey and/or acid whey and/or milk serum.

The invention also provides the use of an enzyme which can exert a deamidating effect on an amide group of a protein, preferably a milk protein, to enhance heat stability of said protein. Furthermore, it provides the use such enzyme to enhance the nutritional value of a powdered food product comprising milk protein, for example a spray dried infant formula.

### LEGEND TO THE FIGURES

FIG. 1: Schematic representation of enzymatic deamidation reaction.

FIG. 2: Isoelectric focusing (IEF) gel electrophoresis of whey protein concentrate treated with glutaminase for 0, 0.5 or 4 hours. Right hand lane indicates pI markers. For details see Example 1.

FIG. 3: Heat stability at 120° C. of whey protein concentrate treated with glutaminase for 0, 0.5 or 4 hours. X-axis indicates the pH at which the concentrate was heated. Y-axis denotes the time (min) at which the first signs of turbidity were observed. See also Example 1.

FIG. 4: Heat stability at 120° C. of an infant formula comprising Hiprotal whey protein concentrate treated with glutaminase for 0, 0.5 or 4 hours, and skim milk powder (SMP). X-axis indicates the pH at which the concentrate was heated. Y-axis denotes the time (min) at which the first signs of turbidity were observed. See also Example 2.

FIG. 5: Effect of heat denaturation on enzymatic deamidation. Whey protein concentrate was left untreated (native) or heated at 85° C. during 1 or 10 minutes prior to incubation with protein glutaminase (E:S ratio=1:10, incubation time 1 h at 40° C., pH 6.5). The degree of deamidation was determined by measuring ammonia release. See also Example 3.

FIG. 6: IEF gel electrophoresis of native (N), preheated at 85° C. for 1 min (1) and 10 min (10), Hiprotal 80BL before and after treatment (+enzyme) with protein glutaminase. M denotes pI markers.

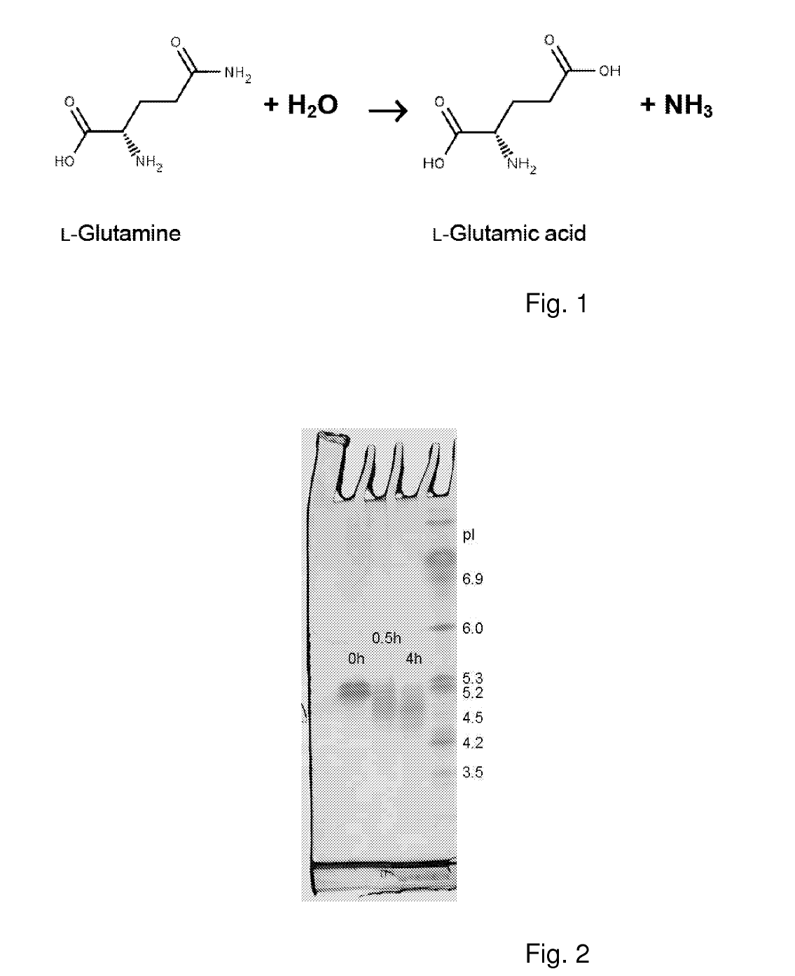

The following non-limiting examples illustrate the invention and do not limit its scope in any way.

### EXAMPLE 1

Whey protein concentrate Hiprotal 80BL, a product with about 80% whey protein on dry matter, derived from cheese whey, was obtained from Friesland Foods Domo (Beilen). The whey protein concentrate was dissolved in demineralized water to a protein content of 2% (w/v) in a non-buffered solution and the pH of the solution was adjusted to pH 6.5 with HCl. The solutions were incubated at 40° C. for 0, 0.5 and 4 hours with Protein Glutaminase ‘Amano’ 500 from Amano Enzyme Inc. (Japan). The enzyme/substrate ratio E:S equaled 1:100. The reaction was stopped/slowed down by immediate and fast cooling to 4-5° C. on ice-water. Samples were stored at 4° C. until use for evaluation of their properties. The protein solutions were characterized on their degree of deamidation by means of ammonia release (see table 1 below) and by means of IEF (FIG. 2).

TABLE 1

| Reaction time [hours] | Gln conversion [mol %] |
|---|---|
| 0.5 | 21 |
| 4 | 55 |

Then, whey protein solutions were adjusted to an ionic strength (I) of 28 mM and a Ca activity of 0.45. Heat stability tests were carried out at 120° C. for a maximum residence time of 20 min with solution-pH ranging from 6.5-7.1 (0.1 pH increment) (subjective method, essentially according to Davies & White, 1966, J. Dairy Res. 33 (1966) 67-81). The pH was varied because pH is known to be an important factor for heat stability. In food products comprising milk protein, the pH often ranges between 6.5 and 7.1.

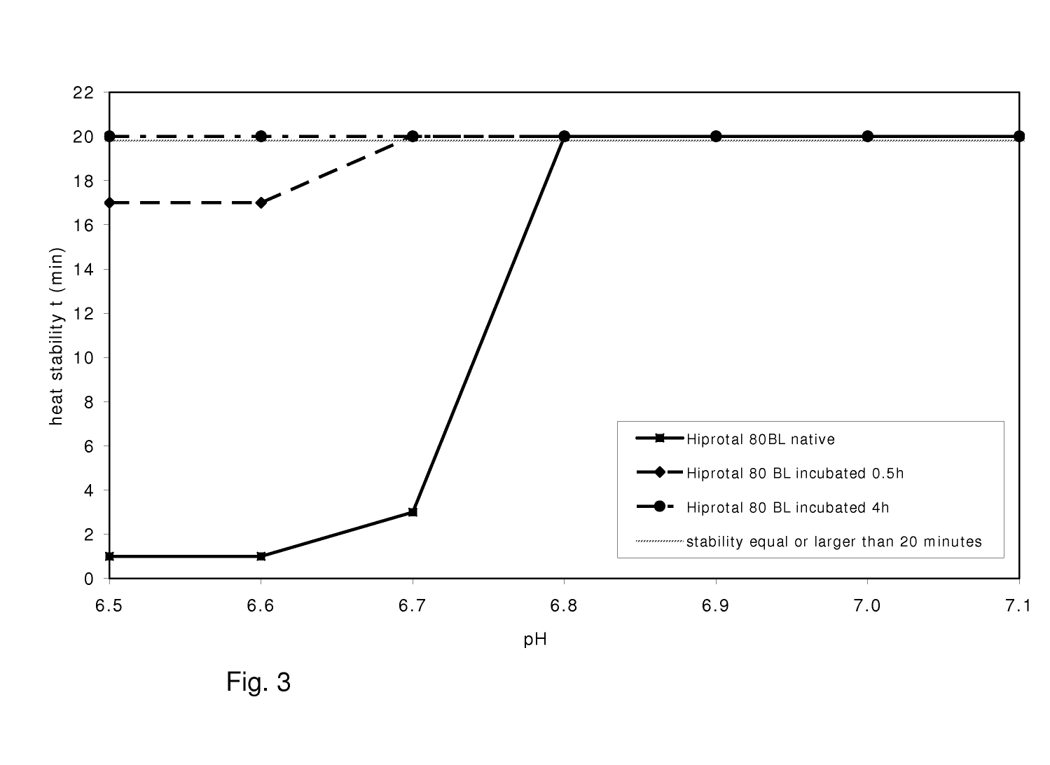

Heat stability was evaluated by eye. Whenever first turbidity (following solution-whitening) was observed, i.e. protein flocs or first aggregates, time (t) was noted. Results as indicated in FIG. 3 demonstrate that enzymatically deamidated Hiprotal 80BL gives improved heat-stability over the native Hiprotal 80BL.

### EXAMPLE 2

An infant formula was prepared by mixing the enzymatically treated whey protein solutions from Example 1 with skim milk powder (SMP) Nilac to obtain a total protein concentration of approximately 1.3% (w/w) in solution with approximately 0.8 and 0.5% (w/w) protein from skim milk and whey protein respectively. The solution was standardized to an ionic strength of I equaling 23 mM and αCa²⁺ activity of 1.7. Heat stability tests were performed at 120° C. for a maximum residence time of 20 minutes, with solution-pH ranging from 6.5-7.1 (0.1 pH increment).

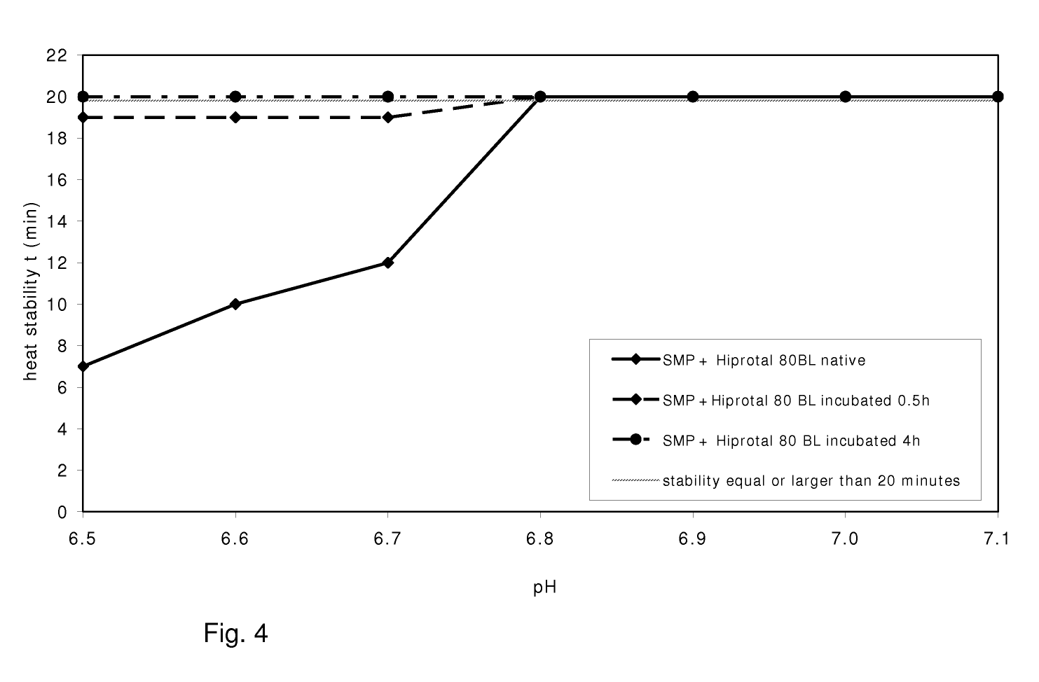

The infant formula with enzymatically treated whey protein shows an overall better heat stability performance (FIG. 4).

### EXAMPLE 3

Whey protein concentrate Hiprotal 80BL, a product with about 80% whey protein on dry matter, derived from cheese whey, was obtained from Friesland Foods Domo (Beilen), β-lactoglobulin A from Sigma Aldrich. Protein glutaminase ‘Amano’ 500 was from Amano Enzyme Inc. (Japan). Hiprotal 80 BL was dissolved in demineralized water to a protein content of 2% (w/v) and the pH of the solution was adjusted to pH 6.5 with HCl. The solutions were incubated at 40° C. for 1.0 h with an enzyme/substrate ratio E:S equaling 1:10, and cooled afterwards to 4-5° C. on ice-water. Two samples were heated before enzyme incubation (1 or 10 min at 85° C.) as to study the effect of heat denaturation on the enzymatic reaction.

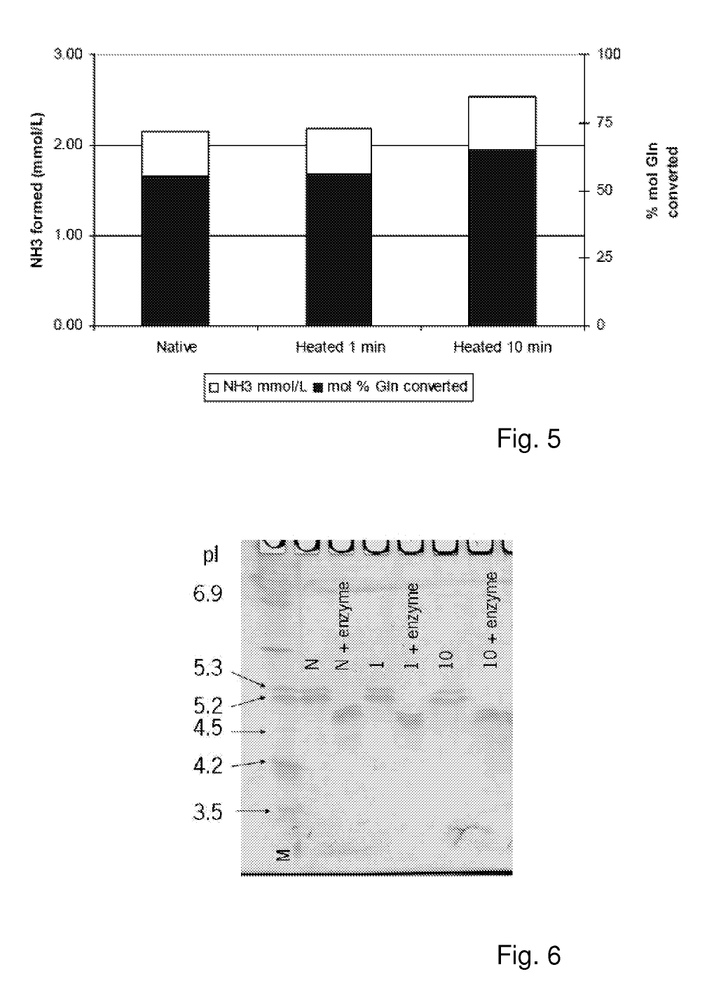

The protein solutions were characterized on their degree of deamidation and by means of IEF (FIG. 5). It can be seen that the effects of heat denaturation are only minor, such that both unheated and heated whey protein are good substrate for the enzyme.

### EXAMPLE 4

Sodium caseinate was obtained from either Sigma Aldrich (C-8654) or Barentz Ingredients (commercial sodium caseinate). Protein glutaminase ‘Amano’ 500 was from Amano Enzyme Inc. (Japan). Sodium caseinate was dissolved in demineralized water to a protein content of 1% (w/v) and the pH of the solution was adjusted to pH 6.5 with HCl. The solutions were incubated at 40° C. for 0.5 and 4 h with an enzyme/substrate ratio E:S equaling 1:200, and cooled afterwards to 4-5° C. on ice-water. The protein solutions were characterized on their degree of deamidation by means of ammonia release [mmol/L] (see table 2 below). Results show that caseinate is easily deamidated.

TABLE 2

Incubation time

| | 0.5 h | 4 h |
|---|---|---|
| Sodium caseinate Sigma | 0.69 ± 0.09 | 2.91 ± 0.08 |
| Commercial sodium | 0.69 ± 0.04 | 3.07 ± 0.05 |
| caseinate | | |

1. A method for the production of a food product comprising a milk protein, comprising subjecting said milk protein to an enzymatic deamidation procedure, formulating the deamidated milk protein into a liquid food product, followed by either heat sterilization of the food product, wherein the temperature during said heat sterilization is at least 110° C., or concentration and spray drying of the food product into a powder.

2. Method according to claim 1, wherein the enzymatic deamidation of said milk protein is carried out with a protein glutaminase.

3. Method according to claim 2, wherein said protein glutaminase is obtained from Chryseobacterium proteolyticum sp No. 6790.

4. Method according to claim 1, wherein the average molecular weight of the milk protein changes less than 10% during enzymatic deamidation.

5. Method according to claim 1, wherein the milk protein which is subjected to an enzymatic deamidation is a protein from cheese whey and/or acid whey and/or milk serum.

6. Method according to claim 1, wherein formulating the liquid food product comprises the preparation of an emulsion and/or evaporation and/or membrane filtration.

7. Method according to claim 1, wherein the temperature during said heat sterilization is at least 115° C.

8. A food product obtainable by a process according to claim 1.

9. Food product according to claim 8, comprising milk protein in which the content of glutamate and/or aspartate residues is higher than in the native protein.

10. Food product according to claim 8, which is a heat sterilized liquid food product.

11. Heat sterilized liquid food product according to claim 10 which is an infant food, a clinical nutritional formula, a creamer, an evaporated milk or a sweetened condensed milk.

12. Heat sterilized liquid food product according to claim 10, wherein the liquid product has a pH up to 6.8, more preferably up to 6.6.

13. Food product according to claim 8, which is a powdered food product, preferably a powdered infant formula or a powdered clinical formula.

14. Food product according to claim 13, wherein the pH of the product dissolved at 10% (w/v) in an aqueous solvent does not exceed 6.8, preferably does not exceed 6.6.

15. Use of an enzymatically deamidated milk protein in a heat sterilized liquid food product.

16. Use of an enzymatically deamidated milk protein in a powdered food product.

17. Use of an enzyme which can exert a deamidating effect on an amide group of a protein, preferably a milk protein, to enhance the heat stability of said protein.
